(19) United States
(12) Patent Application Publication
(10) Pub. No.: US 2006/0100537 A1
(43) Pub. Date: May 11, 2006

(54) Spirometer

(76) Inventors: David R. Williams, Chicago, IL (US); Nicole April Wilson, Lombard, IL (US); Kevin Philip Meade, Westmont, IL (US); Hansen A. Mansy, Justice, IL (US)

Correspondence Address: Douglas H. Pauley;Pauley Petersen & Erickson, Suite 365, 2800 West Higgins Road, Hoffman Estates, IL, 60195, US

(21) Appl. No.: 10/978,910

(22) Filed: Nov. 1, 2004

Related U.S. Application Data
(63) Continuation-in-part of application No. 10/115,263, filed on Apr. 3, 2002.

(51) Int. Cl. A61B 5/08

(52) U.S. Cl. 600/538

(57) ABSTRACT

A spirometer for measuring fluid flow, particularly associated with exhalation of respiratory patients. The spirometer of this invention preferably has a fluidic oscillator wherein the fluid oscillates within a chamber of the fluidic oscillator. An oscillation frequency of the fluid flow within the chamber is correlated to a flow rate. A computer is used to process input data, such as data representing frequency of the oscillatory flow within the chamber, to a flow rate passing through the spirometer. The spirometer of this invention may have no moving parts, which results in the need for only a design calibration and no periodic calibrations throughout use of the spirometer.

### BACKGROUND OF THE INVENTION

1. Field of the Invention

This invention relates to a spirometer, particularly a fluidic oscillator spirometer, for measuring respiratory flow rates.

2. Description of Related Art

In the United States, the American Thoracic Society (ATS) sets guidelines and standards for treatment of people with respiratory disease. ATS guidelines suggest that lung function should be monitored regularly for patients with known respiratory disease. Patients use daily home monitoring of peak flow to periodically check respiratory flow.

Patients and doctors use three main types of conventional devices to assess lung function: diagnostic spirometers, monitoring spirometers, and peak flow meters. Diagnostic spirometers, often used in a medical office, provide the most reliable results. However, diagnostic spirometers are relatively expensive and require significant user training for proper operation. Diagnostic spirometers are not portable and often require the user to own a computer to operate the spirometer.

Diagnostic spirometers produce the most accurate results when assessing lung function. However, the cost of a diagnostic spirometer ranges from about $ U.S. 2,000 to about $ U.S. 10,000, and thus are not readily available or practical for daily home use. Also, diagnostic spirometers can become less accurate as respiratory flow rates become relatively low. Patients with respiratory disease often can achieve only relatively low flow rates during exhalation, and thus the diagnostic spirometer operates in a less accurate range.

The diagnostic spirometer uses a pneumotachigraph, in which fluid flows through hundreds of small tubes and the flow rate is determined by measuring a pressure drop across the tubes. In pneumotach spirometers, air that flows through the tubes is moist and often full of mucus debris. The tubes can become clogged with the mucus debris, which further reduces the accuracy of the diagnostic spirometer. Also, such diagnostic spirometers are difficult to clean and sterilize, primarily because they must be disassembled for thorough cleaning.

Diagnostic spirometers require daily calibration of a pressure drop across the pneumotach. The calibration process is time-consuming and awkward.

Monitoring spirometers are relatively new for pulmonary medicine. The corresponding devices are relatively small and thus portable, and more conducive for home monitoring uses. However, monitoring spirometers are less accurate than diagnostic spirometers. Most monitoring spirometers are used to manually record spirometry values which are typically displayed, for example on a relatively small liquid crystal display. Also, manual recording of spirometry values requires diligent compliance on a daily routine. Because home compliance is a significant problem, manually recorded results are often inaccurate and can result in doctors coming to incorrect conclusions about the daily course of a patient's condition.

Most monitoring spirometers simply report spirometry values. A common measurement in lung function testing is Forced Expiratory Volume in one second (FEV_{1.0}), which relates to the volume of air that a patient can forcefully exhale during the first second of exhalation. However, information contained in the FEV_{1.0} value is not as useful to the physician as a graph of the time-volume curve for each day. The time-volume curve can convey to the physician the nature of the disease but in contrast, a simple number value cannot convey such information. Most diagnostic spirometers produce a time-volume curve but most monitoring spirometers do not produce a time-volume curve.

Conventional peak flow meters can be used to assess lung function. Peak flow meters are relatively inexpensive, portable devices that set the current standard for home monitoring. Peak flow meters measure only a maximum flow rate that a patient can achieve during forceful exhalation. The maximum flow rate measurement provides relatively little useful diagnostic information. However, some physicians believe that because diagnostic results obtained using a measure of peak flow rate are not worth the time and effort involved, patients may avoid use of peak flow meters when performing daily tests.

Some pulmonary physicians believe that daily monitoring of lung function is potentially as beneficial to individuals with lung disease as daily monitoring of blood sugar levels is to individuals with diabetes mellitus, particularly if the respiratory monitoring device can provide diagnostically useful information in a reliable form. It is apparent that there is a need for a spirometer that is relatively small, portable, inexpensive and that can accurately measure, process and record respiratory flow rates.

### SUMMARY OF THE INVENTION

It is one object of this invention to provide a spirometer that is relatively small and can be used as handheld device, particularly in a home environment.

It is another object of this invention to provide a spirometer that uses a fluidic oscillator to measure respiratory flow rates.

It is another object of this invention to provide a spirometer that measures and records predetermined data that a physician can analyze to diagnose lung function.

It is still another object of this invention to provide a spirometer that has no moving parts and that requires no frequent calibration.

The above and other objects of this invention are accomplished with a spirometer that operates with a fluidic oscillator. The spirometer of this invention measures a range of parameters, including Forced Vital Capacity (FVC), which is the amount of air a person can forcefully exhale and including FEV_{1.0}. These particular measurements are significantly more valuable than peak flow measurements, for both diagnostic and monitoring purposes. The spirometer of this invention can electronically record and calculate all measurements. Recordings are stored locally on the device and data can later be transferred to another source, such as a personal computer.

The spirometer of this invention is relatively small and portable, and can be easily and accurately used in a home environment. With the spirometer of this invention, patients can self-monitor between visits to the doctor. The spirometer of this invention eliminates the need for manual recording of respiratory or pulmonary data received as a result of daily monitoring.

### BRIEF DESCRIPTION OF THE DRAWINGS

The above and other features of this invention are apparent when this specification is read in view of the drawings, wherein:

FIG. 1 is a perspective view of one half of an oscillatory flow spirometer, cut along and symmetric about a centerline, according to one embodiment of this invention;

FIG. 1A is a sectional view along a centerline of the oscillatory flow spirometer, as shown in FIG. 1;

FIG. 1B is a sectional view along a centerline of an oscillatory flow spirometer, similar to the embodiment shown in FIGS. 1 and 1A but having a gap between wedge elements;

FIG. 1C is a schematic diagram showing a height and width of a nozzle, according to one embodiment of this invention;

FIG. 2 is an electronics system diagram showing operation of a computer or an electronics package associated with the spirometer of this invention;

FIG. 3 is a graph showing flow rate versus frequency for a fluidic oscillator, according to one embodiment of this invention;

FIG. 4 is a graph of pressure drop versus flow rate, wherein the solid line represents a maximum allowable pressure drop according to monitoring standards of the American Thoracic Society Standardization of Spirometry 1994 Update;

FIG. 4A is a graph of pressure drop versus flow rate, wherein the solid line represents a maximum allowable pressure drop according to diagnostic standards of the American Thoracic Society Standardization of Spirometry 1994 Update;

FIG. 5 is a graph illustrating a linear frequency response of a fluidic oscillator, according to one preferred embodiment of this invention;

FIG. 6 is a schematic diagram of a differential amplifier and a zero crossing detector, according to one embodiment of this invention;

FIG. 7 is a block diagram showing steps of an analog processor and a digital processor, according to one preferred embodiment of this invention;

FIG. 8 is a rear perspective view of a spirometer having a handle detachably mounted with respect to a housing of the spirometer;

FIG. 9 is a front perspective view of the spirometer with the interchangeable handle, as shown in FIG. 8;

FIG. 10 is a sectional view taken along a longitudinal axis of a handle, according to one embodiment of this invention;

FIG. 11 is a sectional view, taken along line 11-11 as shown in FIG. 10, of the handle as shown in FIG. 10; and

FIG. 12 is a perspective view of a spirometer with a handle and with a detachable filter element mouthpiece, according to one embodiment of this invention.

### DESCRIPTION OF PREFERRED EMBODIMENTS

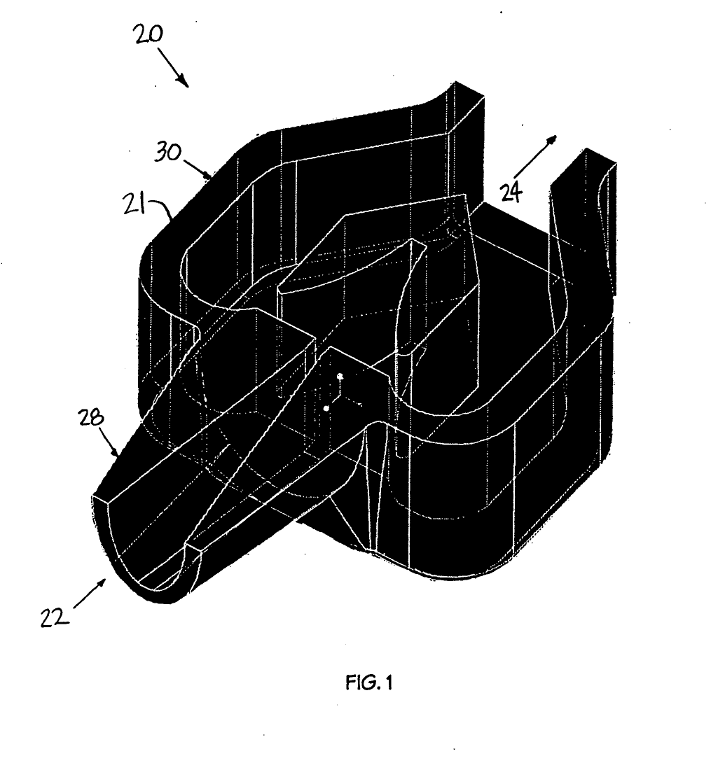

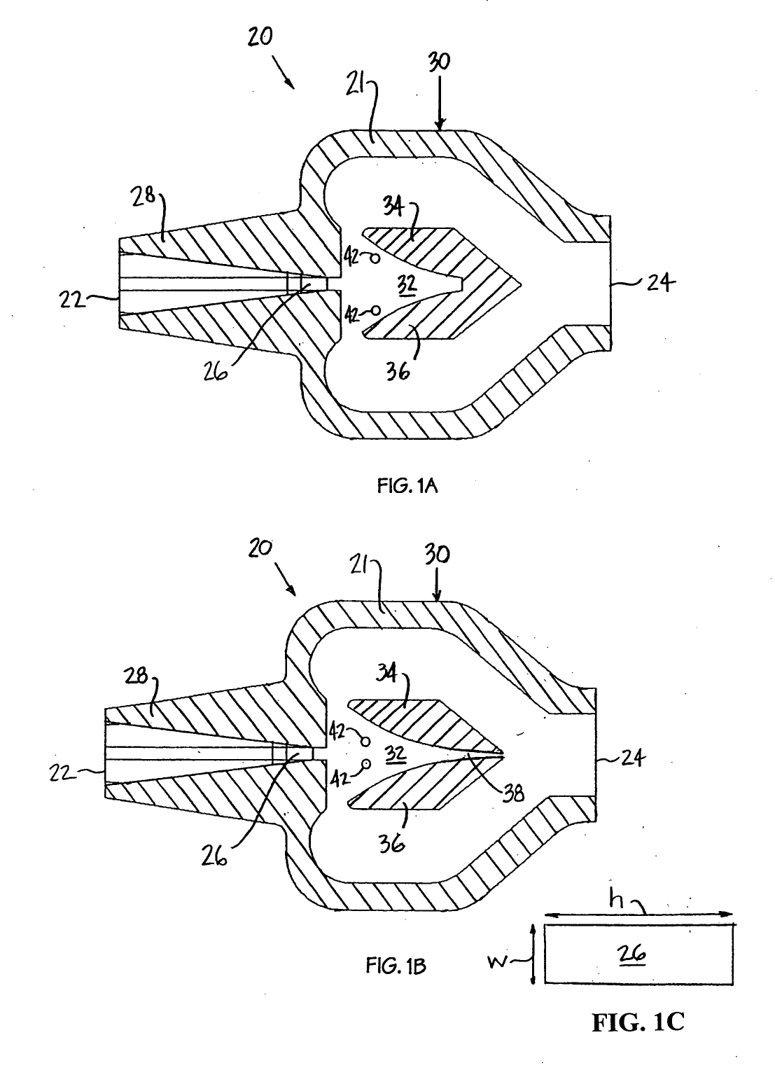

Spirometer 20 of this invention is a relatively small, preferably handheld device that operates using principles of oscillatory flow. Throughout this specification and in the claims, the word fluid is intended to relate to air or the fluidic content of an exhalation discharge from a patient, or any other similar fluid. The fluid enters spirometer 20 through inlet 22, and is ultimately discharged through outlet 24, as shown in FIGS. 1, 1A and 1B.

Mouthpiece 28 can be mounted directly or indirectly with respect to nozzle 26, so that the fluid flows through mouthpiece 28, through nozzle 26 and also through inlet 22.

According to one embodiment of this invention, spirometer 20 comprises a fluidic oscillator flowmeter. Conventional fluidic oscillator devices exist. For example, U.S. Pat. Nos. 4,843,889 and 5,363,704, the teachings of which are incorporated into the specification by reference to both United States Patents, teach a fluidic oscillator, for example one that can be used as fluidic oscillator 30 of this invention.

Fluidic oscillator 30 of this invention comprises chamber 32. In a used condition of spirometer 20, where fluid flows through spirometer 20, the fluid oscillates within chamber 32. As shown in FIG. 1, wedge elements 34 and 36 are mounted within chamber 32. In one embodiment of this invention, fluid flows into chamber 32 and impinges or otherwise contacts wedge elements 34 and 36. The shape, size and/or position of each wedge element 34, 36 can be varied to accomplish different oscillatory fluid flow parameters.

Depending on the shape of chamber 32 and the particular layout, size and/or shape of each wedge element 34, 36, spirometer 20 can be calibrated as a function of predetermined design. In one embodiment of this invention, fluidic oscillator 30 has no moving parts. In another embodiment of this invention spirometer 20, including all elements, has no moving parts. Without moving parts, spirometer 20 can be accurately calibrated initially and require no later periodic calibration.

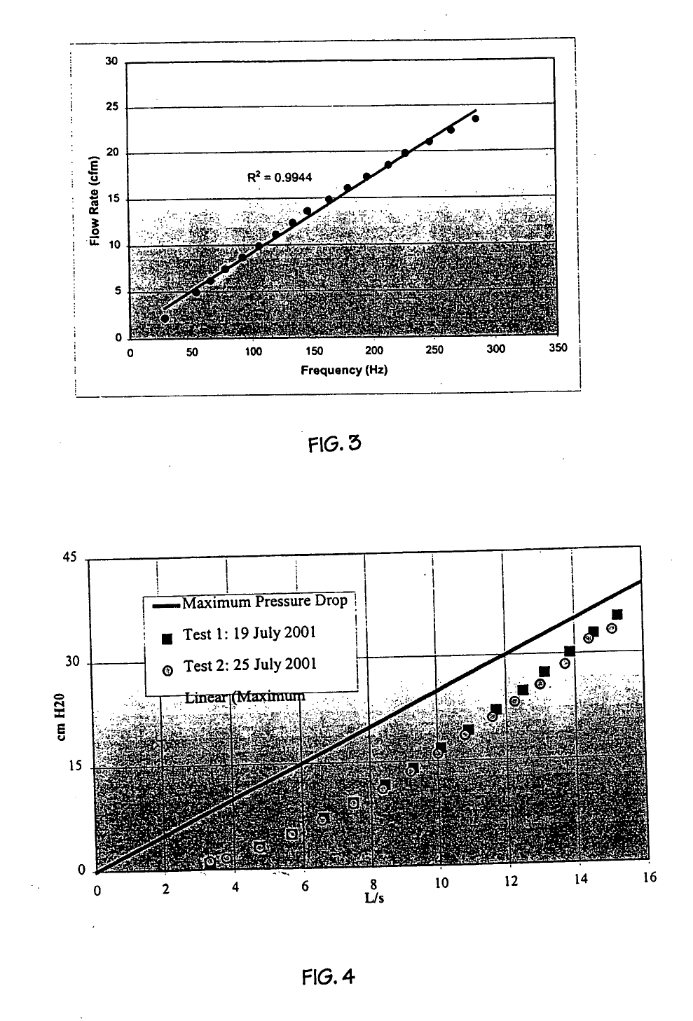

In one embodiment of this invention, oscillation frequency in chamber 32 is linearly proportional to the flow rate of the fluid entering through inlet 22. FIG. 3 shows a graph of oscillation frequency versus flow rate. The frequency of oscillation can be linearly correlated to flow rate. R-squared values can be determined using a least squares regression technique, such as known to those skilled in the art of mathematics.

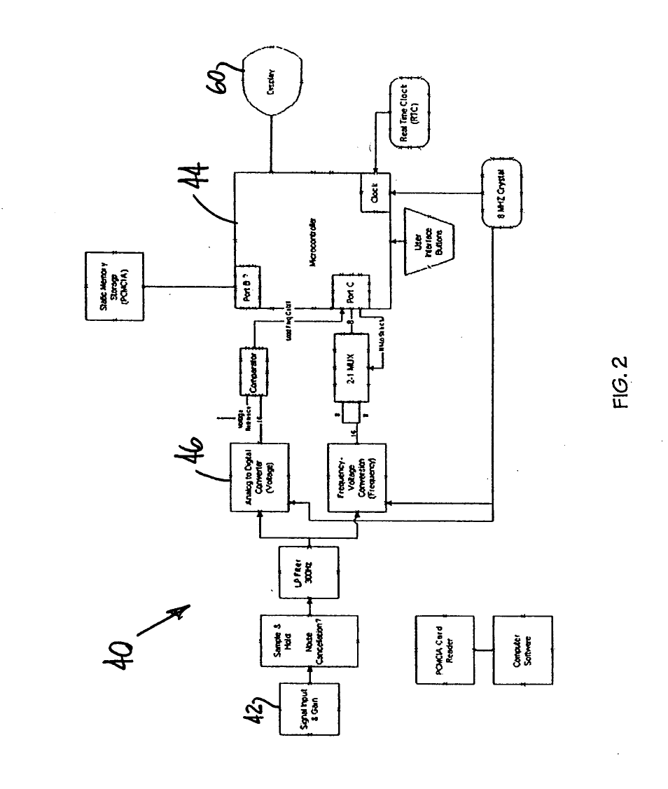

In one embodiment of this invention, computer 40, as shown in FIG. 2, is used to measure the oscillatory frequency and then to calculate a standard spirometry value or values and one or more time-volume curves. Computer 40 can comprise any suitable processing device mounted within any suitable frame or other hardware, such as known to those skilled in the art of computers. The hardware can be mounted directly to or with respect to housing 21 of spirometer 20.

A processor of computer 40 can be designed specifically for spirometer 20 of this invention, and can include an analog sensing circuit, with sensor 42, such as an integrated thermistor or pressure transducer, for sensing fluidic oscillations. The processing unit may also comprise a 16-bit analog-to-digital conversion unit with parallel output, a frequency-to-voltage convertor, a microcontroller, and flash memory cards or another suitable digital data storage device.

Sensor 42 detects pressure fluctuations and correlates detected data to an oscillation frequency.

Fluidic oscillation can be varied by selecting a position of wedge elements 34 and 36 with respect to each other. In one preferred embodiment, wedge element 34 contacts wedge element 36. In another embodiment, wedge element 34 is integrated as one piece with wedge element 36. In another embodiment of this invention, such as shown in FIG. 1B, gap 38 is defined between wedge element 34 and wedge element 36, or as disclosed in U.S. Pat. No. 4,843,889.

Sensor 42 can send an input signal, either analog or digital, to the microcontroller of computer 40. The input signal can be transmitted as an analog signal to the microcontroller and then converted to a digital signal or can be converted to a digital signal locally at sensor 42 and then transmitted to the microcontroller.

In one embodiment of this invention, the microcontroller can be programmed or loaded with a suitable algorithium that corresponds to particular data, such as the data as shown in FIG. 3. The microcontroller can then process input data and produce an output signal which can be delivered to output device 60. Output device 60 may comprise any suitable hardware, such as a monitor or other readout display, mounted with respect to housing 21 of spirometer 20.

Computer 40 can provide an interface between frequency and/or flow rate information obtained from chamber 32 and the resultant volumetric flow measurements. In one embodiment of this invention, sensor 42 comprises a thermistor sensing the fluidic oscillations and a processor which calculates and determines the FVC and FEV_{1.0}, and can store results as calculated values and/or arithmetic equations.

In one embodiment of this invention, computer 40 calculates and determines the flow rate through spirometer 20 as a function, such as a directly proportional function, of an oscillation frequency of the fluid passing through chamber 32.

In one embodiment of this invention, the oscillation frequency is in a range from about 0 Hz to about 400 Hz, but depending upon the design of chamber 32 the oscillation frequency can be higher. According to one embodiment of this invention, it is only necessary to measure the oscillation frequency to determine the flow rate. Once spirometer 20 of this invention is calibrated for a particular design, it is not necessary to measure pressure drops across any one or more elements of spirometer 20. Sensor 42 produces an output signal which is eventually converted to an electrical signal. The electrical signal is preferably amplified and/or further processed.

An electronics package can be used to detect vortex oscillations occurring within fluidic oscillator 30, such as a trapped-vortex air fluidic oscillator. A pressure sensor, such as an analog pressure sensor, can be used to measure vortex oscillation periods, which can be digitally recorded by an analog-to-digital convertor and microprocessor 44. Each vortex oscillation corresponds to a specific volume of fluid moving through spirometer 20, which can be determined by a static calibration. The total volume of fluid passing through fluidic oscillator 30 can be measured by counting a number of vortex oscillation cycles. In one embodiment of this invention, the flow rate as a function of time is determined from a period⁻¹ vs. time plot.

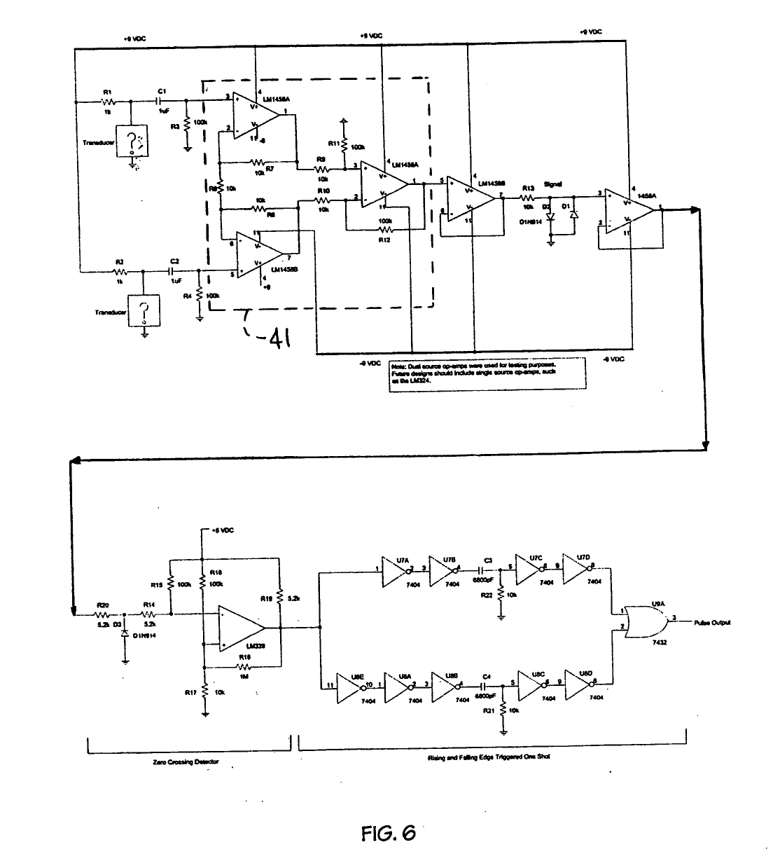

FIG. 6 shows one embodiment of an electrical circuit that can be used as part of computer 40. The dashed lines shown in FIG. 6 identify differential amplifier circuit 41, the type shown and other types of which are known to those skilled in the art of electronic circuits. Many different operational amplifiers, filters and/or buffers can be used to process the output signal emitted by sensor 42.

In one embodiment of this invention, a zero crossing detector, which operates as a function of a voltage magnitude of an electrical signal that alternates between a positive maximum and a negative maximum about a reference voltage, can be used to identify the oscillation frequency. A voltage comparator, such as an operational amplifier device that compares voltages at input terminals, can also be used as part of the zero crossing detector. FIG. 6 shows one embodiment of a zero crossing detector that can be used with computer 40 of this invention.

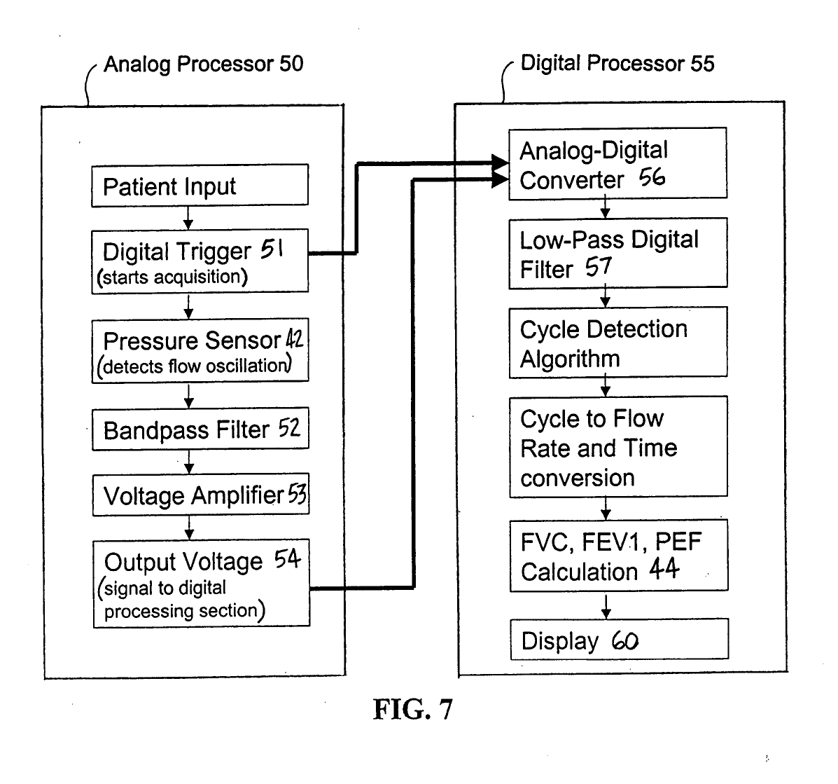

In one embodiment of this invention, signal processing includes two components, analog processor 50 and digital processor 55, such as shown in FIG. 7. In one embodiment of this invention, analog processor 50 comprises an on/off switch, such as digital trigger 51. As shown in FIG. 7, trigger 51 signals pressure sensor 42 to detect a flow oscillation and send a signal to bandpass filter 52, such as a Butterworth filter, which emits a signal to voltage amplifier 53. Output voltage 54, which can be accomplished with a battery power supply, can be emitted as an analog signal to digital processor 55. Analog processor 50 converts the analog pressure fluctuations within fluidic oscillator 30 to a time-varying voltage signal suitable to be received by digital processor 55.

As shown in FIG. 7, digital processor 55 comprises Analog-Digital convertor 56 that receives output voltage 54 and converts the analog signal to a digital signal which is passed on to low-pass digital filter 57. A digital signal is passed from low-pass digital filter 57 to microcontroller 44, which records and stores a time series of voltages from analog processor 50. Software associated with microcontroller 44 includes an algorithm that converts a time series of pressure sensor data into a flow rate. The flow rate data is converted into values, such as for vital capacity, peak flow rate, forced expiratory volume after a specified time period, such as one second, as well as other values required by ATS standards for a spirometer.

The calculated values can be determined from output device 60, such as a digital display. In one embodiment of this invention, a laptop or desktop computer and/or a PDA device can be used as microcontroller 44 and/or display 60.

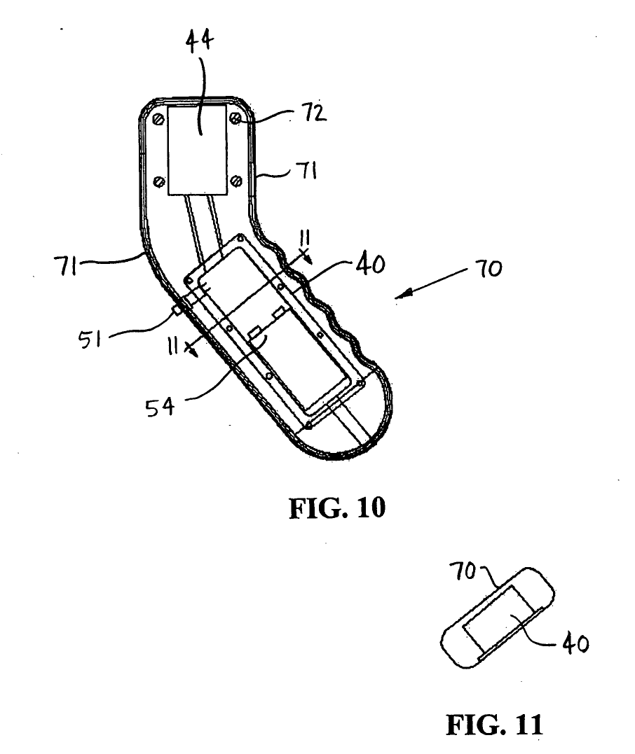

In one embodiment of this invention, hardware associated with analog processor 50 can fit within interchangeable handle 70, such as shown in FIGS. 10-12, for holding spirometer 20.

In one embodiment of this invention, a method for determining an exhalation flow rate of a respiratory system uses spirometer 20 of this invention. Fluid flow is directed into nozzle 26 and passed through inlet 22, into chamber 32 of fluidic oscillator 30. The fluid flow oscillates within chamber 32 and an oscillation frequency of the fluid flow is detected within chamber 32. An input signal representing an oscillation frequency within chamber 32 is detected and delivered to computer 40, which then processes the input signal and emits an output signal. The output signal correlates a flow rate of the fluid flow, which is preferably but not necessarily linearly proportional to the oscillation frequency. In one embodiment of this invention, a least squares regression analysis is used to calibrate, such as initially, spirometer 20 and a resulting linear equation is used to calculate the flow rate as a function of the oscillation frequency. The output signal can be delivered to an output device and displayed for reading purposes, or can be further delivered to another electronic device for further signal processing.

Spirometer 20 of this invention can be used to determine and process volumetric flow data which can be useful in pulmonary medicine. Spirometer 20 of this invention can be designed and calibrated to conform to guidelines set by the American Thoracic Society (ATS). ATS guidelines require a specific pressure drop across the flow meter and spirometer 20 of this invention can be designed to meet any such specific pressure drop requirement.

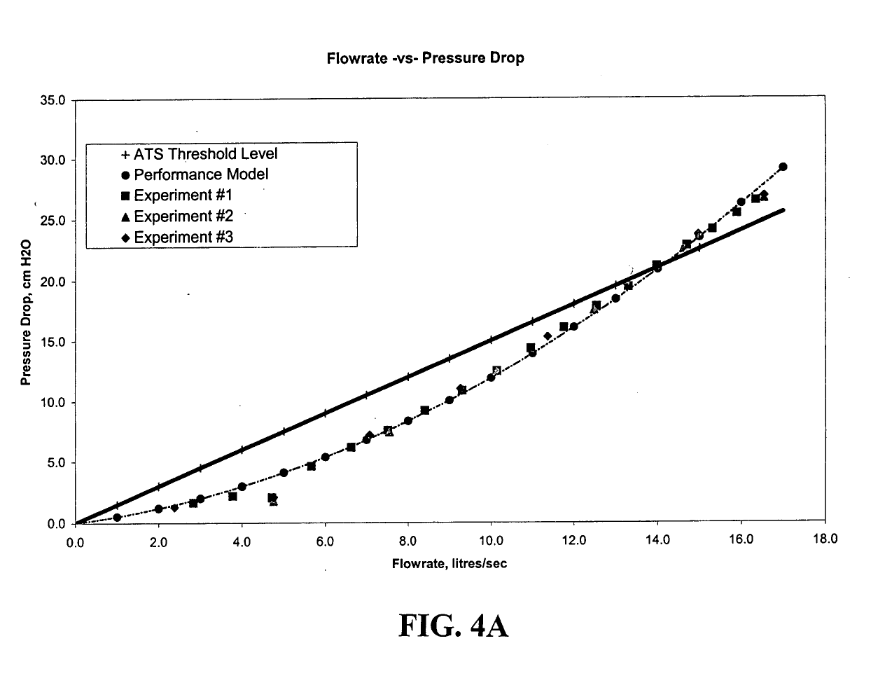

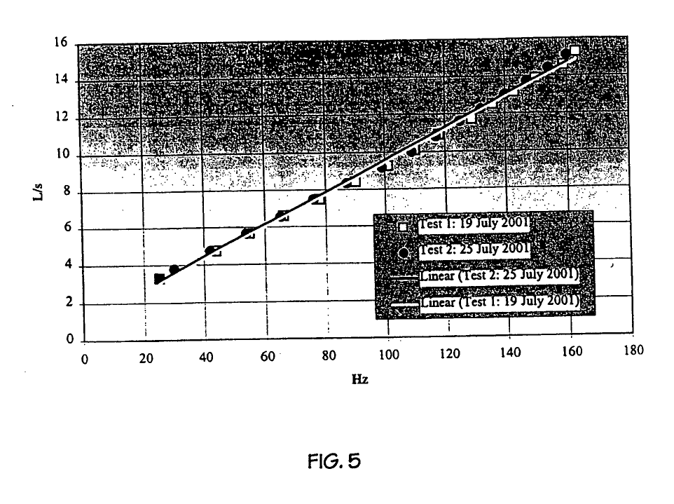

ATS guidelines may also require the nozzle of a spirometer to have a specific pressure drop, depending upon whether the spirometer is used for monitoring purposes or diagnostic purposes. Nozzle 26 of this invention can be designed to meet any such specific pressure drop requirement. For example, the solid line in FIG. 4A shows ATS guidelines that require specific pressure drops for diagnostic spirometry. As shown in FIG. 4A, in a flow range from zero to 14 L/s, the resistance and back pressure of the diagnostic spirometer must be less than 1.5 cm H₂O/L/s, according to ATS guidelines. Thus, at 14 L/s, the maximum allowable pressure drop across the diagnostic spirometer is 21.0 cm H₂O. According to spirometer 20 of this invention, the flow range and pressure drop requirements for a diagnostic spirometer can be met by using a nozzle designed having an aspect ratio in a range from about 20 to about 160, wherein the aspect ratio is defined as the height of the nozzle divided by the width of the nozzle. The solid line in FIG. 4 shows ATS guidelines that require specific pressure drops for a monitoring spirometer which in a flow range from zero to 14 L/s must have a back pressure of the monitoring spirometer which is less than 2.5 cm H₂O/L/s. The curved line below the solid line in each of FIGS. 4 and 4A shows data points supporting that the overall pressure drop across spirometer 20 of this invention can be less than the maximum pressure drop allowed, according to ATS guidelines, in a flow range from zero to 14 L/s.

In preferred embodiments of this invention, nozzle 26 can have a height dimension in a range from about 6 cm to about 21 cm, and can have a width dimension in a range from about 0.13 cm to about 0.40 cm. Preferably but not necessarily, an aspect ratio is selected so that the width dimension of nozzle 26 provides practical and easy use of spirometer 20. In one embodiment of this invention, nozzle 26 is 0.36 cm in height by 7.4 cm in width, with an aspect ratio of about 20.5, and thus a fluid jet at nozzle 26 has similar dimensions.

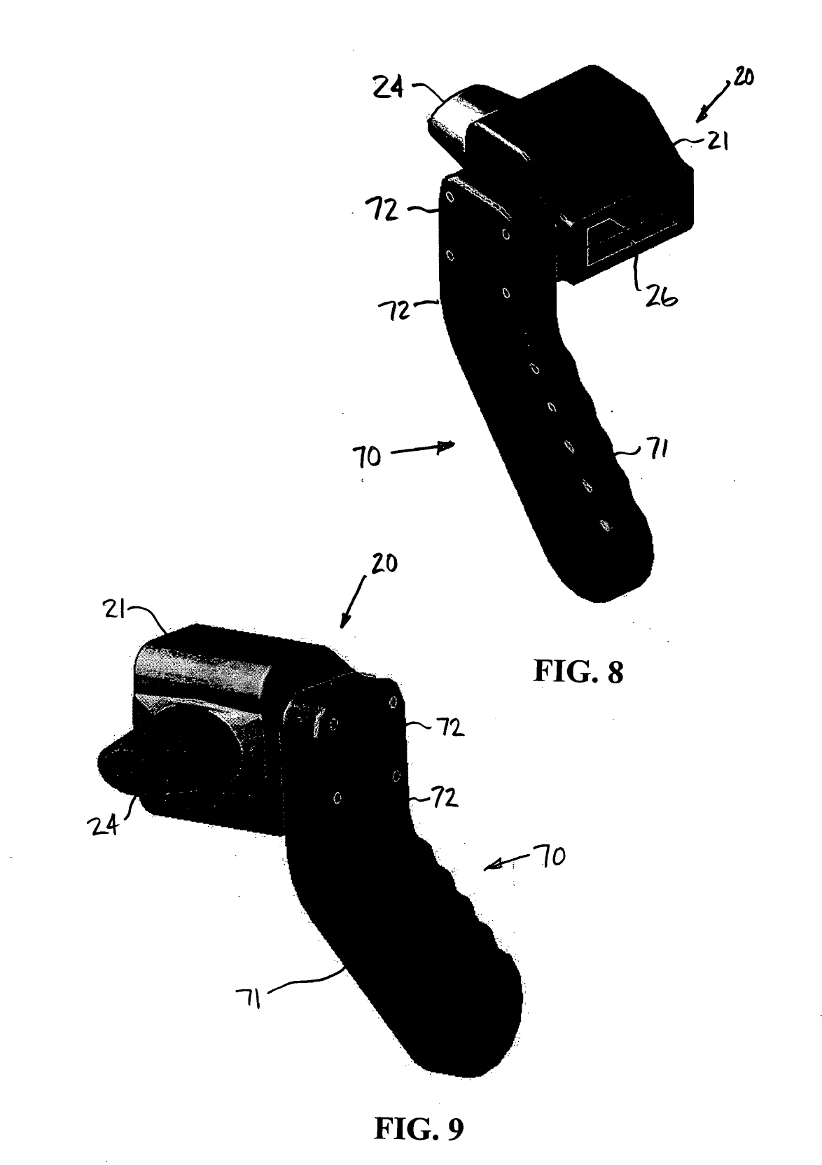

FIGS. 8 and 9 show handle 70, according to one embodiment of this invention. Handle 70 can be detachably attached with respect to housing 21 of spirometer 20. An interchangeable handle 70 can be used to clean and/or replace housing 21 and other elements associated with spirometer 20, for example to prevent cross-contamination between patients or users of spirometer 20.

As shown in FIGS. 10- 12, the electronic components of analog processor 50, digital processor 55, microprocessor 44 and/or computer 40 can be mounted within housing 71 of handle 70. Also with handle 70 being interchangeable with respect to housing 21, fluidic oscillator 30 and its associated elements can be cleaned without causing moisture damage to the electronics components. As shown in FIGS. 8-10, connecting pins 72 are used to detachably attach housing 21 with respect to handle 70. Any other suitable mechanical connection can be used to detachably attach handle 70 with respect to housing 21.

Mouthpiece 28 can be attached directly or indirectly to nozzle 26. The design of mouthpiece 28 is selected to structurally conform with and correspond to nozzle 26, and so that particular flow parameters are achieved through mouthpiece 28 and nozzle 26, for entry into inlet 22. Mouthpiece 28 preferably fits comfortably within a patient's mouth.

In one embodiment of this invention, a bacterial and/or viral filter element can be inserted between handle 70 and housing 21. The filter element can be sandwiched within a recess formed between handle 70 and housing 21, when in an assembled condition. The filter element can be of a sheet material, such as a sheet of filtering media, or can be any other suitable filter.

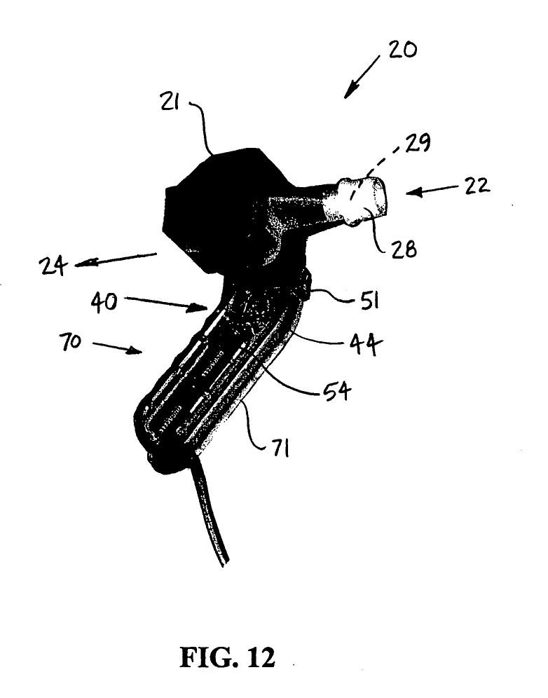

FIG. 12 also shows mouthpiece filter 29 which can be used in combination with or in lieu of filter element 80, so that the entire exhalation flow is filtered. Mouthpiece filter 29 may comprise any filtering media sheet or other suitable filter mounted within mouthpiece 28. For example, mouthpiece 28 may comprise two or more separable portions that can be separated to position or replace mouthpiece filter 29. One or more portions of mouthpiece 28 can be disposable or can be sanitized after direct contact with a patient. Any portion downstream of mouthpiece filter 29 may or may not be disposable or sanitizable. Any filter media is preferably but not necessarily changed between patients or daily, if used by the same patient, and preferably but not necessarily has a filtering efficiency of about 99.9 percent. However, any other efficiency or filter media shape or content, and/or filter mounting can be used without departing from this invention.

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

We claim:

1. An apparatus for determining an entire exhalation flow rate of a respiratory system, the apparatus comprising:
a spirometer having an inlet for accepting the entire exhalation flow rate and having an outlet, a fluidic flow oscillator in communication with the inlet and the outlet, a nozzle in communication with the inlet, and the nozzle having an aspect ratio in a range from about 20 to about 160.

2. The apparatus according to claim 1, wherein the aspect ratio is defined as a height of an opening of the nozzle divided by a width of the opening of the nozzle.

3. The apparatus according to claim 2, wherein the width is in a range from about 0.13 cm to bout 0.40 cm.

4. The apparatus according to claim 2, wherein the height is in a range from about 6 cm to about 21 cm.

5. The apparatus according to claim 1, wherein an entire exhalation fluid flow from the nozzle is passed into and through the spirometer at a maximum pressure drop across the spirometer that is less than 1.5 cmH₂O per L/s between a flow rate of zero and 14 L/s.

6. The apparatus according to claim 1, further comprising a computer, a sensor positioned within a chamber of the fluidic flow oscillator, the sensor detecting an oscillation frequency of the fluid flow within the chamber and emitting a corresponding input signal to the computer.

7. The apparatus according to claim 6 wherein the sensor comprises an analog sensing circuit that emits the input signal as an analog signal, and the computer comprises a microcontroller and a convertor that receives and converts the analog signal to a digital signal for the microcontroller to process.

8. The apparatus according to claim 6, further comprising a handle detachably mounted with respect to a body of the fluidic oscillator flowmeter.

9. The apparatus according to claim 8, wherein the microcontroller is housed within a housing of the handle.

10. The apparatus according to claim 1, further comprising a mouthpiece detachably mounted with respect to a body of the fluidic oscillator flowmeter and in communication with the nozzle, and a filter element replaceably mounted within the mouthpiece.

11. An apparatus for determining an entire exhalation flow rate of a respiratory system, the apparatus comprising:
a spirometer having an inlet for accepting the entire exhalation flow rate and having an outlet, a fluidic flow oscillator in communication with the inlet and the outlet, and during the entire exhalation flow rate a maximum pressure drop across the spirometer being less than 1.5 cm H₂O per L/s between a flow rate of zero and 14 L/s.

12. A method for determining an exhalation flow rate of a respiratory system, the method comprising:
discharging an entire exhalation fluid flow into a nozzle of a spirometer, and passing the entire exhalation fluid flow through the nozzle that has an aspect ratio in a range from about 20 to about 160 and through a fluidic oscillator flowmeter.

13. The method according to claim 12, wherein the aspect ratio is defined as a height of an opening of the nozzle divided by a width of the opening of the nozzle.

14. The method according to claim 13, wherein the width is in a range from about 0.13 cm to bout 0.40 cm.

15. The method according to claim 13, wherein the height is in a range from about 6 cm to about 21 cm.

16. The method according to claim 12, wherein an entire exhalation fluid flow from the nozzle is passed into and through the spirometer at a maximum pressure drop across the spirometer that is less than 1.5 cmH₂O per L/s between a flow rate of zero and 14 L/s.

17. The method according to claim 12, wherein an input signal representing an oscillation frequency is detected in a chamber of the fluidic oscillator flowmeter and is computed into an output signal.

18. A method for determining an exhalation flow rate of a respiratory system, the method comprising:
discharging an entire exhalation fluid flow into a nozzle of a fluidic flow oscillator of a spirometer, and passing the entire exhalation fluid flow through the spirometer at a maximum pressure drop across the spirometer that is less than 1.5 cm H₂O per L/s between a flow rate of zero and 14 L/s.
